(19) United States
(12) Patent Application Publication
(10) Pub. No.: US 2009/0171404 A1
(43) Pub. Date: Jul. 2, 2009

(54) ENERGY GENERATING SYSTEMS FOR IMPLANTED MEDICAL DEVICES

(75) Inventors: Afraaz Irani, Santa Clara, CA (US); Mark Bianco, Mountain View, CA (US); David Tran, Stanford, CA (US); Peter Daniel Deyoung, Deer Park, IL (US); Melanie Lisa Romola Wyld, Palo Alto, CA (US); Tony Hansheng Li, Mountain View, CA (US)

Correspondence Address: WILSON SONSINI GOODRICH & ROSATI, 650 PAGE MILL ROAD, PALO ALTO, CA, 94304-1050, US

(73) Assignee: LELAND STANDFORD JUNIOR UNIVERSITY, Palo Alto, CA (US)

(21) Appl. No.: 12/293,218

(22) PCT Filed: Mar. 19, 2007
(86) PCT No.: PCT/US07/06917

§ 371 Date: Mar. 10, 2009

Related U.S. Application Data
(60) Provisional application No. 60/782,837, filed on Mar. 17, 2006.

(51) Int. Cl. A61N 1/02; F03G 5/06

(52) U.S. Cl. 607/2; 290/1 R

(57) ABSTRACT

Devices and systems for generating energy for powering implanted medical devices such as a pacemakers and defibrillators.

### RELATIONSHIP TO OTHER APPLICATIONS

This application claims the benefit of and priority to U.S. provisional patent application No. 60/782,837, filed on 17 Mar. 2006 and Titled High Endurance Pacemakers and IDCs, which application is hereby fully incorporated by reference for all purposes.

### BACKGROUND

Currently, a number of “active” implanted medical devices are in use that require an input of power. The power source may be internal or external to the device, and usually consist of electrical-chemical cells (batteries). Examples of common active implanted devices include:

cardiac pacemakers used to treat conduction disorders and heart failure

cardiac defibrillators used to treat ventricular and atrial tachyarrhythmia and fibrillation

left ventricular assist devices used to treat heart failure

muscle stimulators used to treat, for example, urinary incontinence and gastroparesis

neurological stimulators used to treat essential tremor (e.g. due to parkinson's disease)

cochlear implants used to treat hearing disorders

monitoring devices used to treat seizures, for example

drug pumps used to administer drugs, for example to treat pain, diabetes (insulin pumps), spasticity (intrathecal baclofen pumps).

Obviously longevity and reliability are major concerns for the manufacturers (and users) of such implanted devices. Battery failure is the leading cause of re-operation for pacemakers and implantable cardioverter defibrillators (ICDs) and 76% of pacemaker failures are due to battery failure. Re-operations are undesirable because they increase patient complications and add substantial financial expense to health care. There is a need to reduce the incidence of re-operation due to power depletion in implanted pacemakers and ICDs.

### A. Implantable Electrical Devices

### Pacemakers, ICDs, BVPs

Pacemakers and ICDs have similar designs and structures. The main differences between them are size, internal circuitry, and the number of leads. The devices comprise three major components: (1) a generator, (2) a connector, and (3) leads.

The generator includes a battery that powers the device and electronics that monitor the heart's activity and generates electric impulses, all housed within a lightweight, smooth plastic biocompatible casing⁴. These devices use lithium ion batteries. In the past, electromedical devices were powered by nickel-cadmium and mercury-zinc batteries, nuclear (plutonium) power batteries, and at one point even biological batteries⁷. The ICD's generator is larger in size than that of a pacemaker, and the electronics within pacemakers and ICDs are different, since the two devices treat different diseases.

The connector is a plastic head which connects and secures the leads to the generator.

The leads are flexible insulated biocompatible wires that deliver the electric impulses to the heart from the generator. The ICD has more leads than a pacemaker. In a pacemaker, leads are anchored in the right atrium and the right ventricle. Leads sense the beating of the heart and transmit impulses for it to beat faster.

Pacemakers

Pacemakers produce low voltage rhythmic electrical signals that remedy a diseased heart's defective ability to generate its own electrical signals, which may cause the heart to beat to be too fast, too slow, or irregularly. The pacemaker continuously monitors the heart's electrical system, and delivers an electrical impulse to aid the heart when it detects a need for it. The vast majority of pacemakers are used to treat bradyarrhythmia or bradycardia, which is when the heart beats too slowly due to a defect in the sinoatrial node or a blockage in the heart's own electrical conduction system, thus reducing blood flow and prohibiting the body from receiving the blood it needs.

The batteries in pacemakers can last up to ten years, although they typically last four to five years. This is a significant improvement from the first battery powered pacemaker which lasted just 12-18 months.

ICDs

ICDs deliver electrical impulses to the heart when it detects cardiac arrest or other irregular rhythms caused by a heart disease. They are about the twice the size of a pacemaker and are implanted under the skin.

The NIH defines five major groups of candidates who could benefit from an ICD: Those who have survived a cardiac arrest due to VF not triggered by a recent heart attack; Those with life-threatening episodes of VT; survivors of a heart attack with weakened pumping function; those who have structural defects of the heart muscle, such as dilated cardiomyopathy and hypertrophic cardiomyopathy, especially when unexplained fainting episodes have occurred; people with a reduced pumping function of the heart, often assessed as a left ventricular ejection fraction (LVEF) of 35% or less.

BVPs

A BVP is a particular type of pacemaker that is used to deliver cardiac resynchronization therapy to treat patients with congestive heart failure. The additional leads (3 or 4 instead of 2 for a normal pacemaker) allow the pacemaker to ensure that the left and right ventricles fire at the same time. When a patient is suffering from CHF the two ventricles do not always fire at the same time, which reduces the ability of the heart to eject sufficient blood with each contraction.

The implantable device market is very large. It is estimated that in 2005, the overall market size was $9.2 billion. The ICD industry generated the lion's share of this with revenues of $6.2 billion. Pacemakers made up the remaining $3 billion.

### Procedure for Implantation

The basic procedure of implanting pacemakers and ICDs is the same. The pacemaker implantation operation typically lasts from 1-2 hours, while the ICD implantation operation lasts 2-3 hours. First the patient, after undergoing the generic pre-operation routine, has his chest locally anesthetized where the 2 inch incision is to be made. Then the device is calibrated to the patient and the lead(s) are inserted through a 2-4 inch incision in the chest beneath the collarbone, traveling via a vein until it reaches the heart. The lead(s) are then guided and set into their correct positions in the heart. The generator is then placed by the physician between the skin and pectoral muscle and situated into a stable position. In addition, the device is further calibrated to ensure proper operation before the incision is closed. Following surgery, patients are given antibiotics to fight possible infections. Usually the patient will be checked every two weeks for the first month to see if the rate, parameters, etc. of the pacemaker need to be adjusted. Check-ups are performed six months after that and then usually either ever 6 months or every year following that. These regular checkups are also used to assess the life of the battery.

### Re-Operation

Since the devices are self contained they have a limited life span. 76% of pacemaker failures are due to battery failure. When the device fails, surgeons reopen the wound and remove the old device, replacing it with a new one while keeping the original leads in the patient. As with any surgery, there is a risk or re-infection when performing follow-up surgery. Accordingly, if these follow up surgeries could be prevented, risk of infection would be minimized. This would result in a better outcome for the patients while also being cheaper. Complications can occur in any surgery, and re-operation procedures are no different. These surgeries have the following complications and complication rates: Mortality rate of 1%; Pocket Hematoma of 4.9%; Infection 5%; Skin Erosion 7.7%.

The implanted systems described all require some type of power storage device. Various means of power generation, charging, and power storage have been considered. This includes primary chemical batteries of all sorts, nuclear batteries, and rechargeable batteries. Some power systems place the power pack outside the patient's body, with pulses of energy being transmitted to a passive implanted receiver and lead. Rechargeable pacemaker devices may incorporate a charging circuit which is energized by electromagnetic induction, or other means. This produced a current in the charging circuit which flowed to the rechargeable battery. Cardiac pacemakers based on rechargeable batteries are described in art references, including U.S. Pat. Nos. 3,454,012, 3,824,129, 3,867,950, 3,888,260 and 4,014,346. Other relevant publications include the following: U.S. Pat. No. 3,563,245 titled Biologically Implantable and Energized Power Supply, issued Feb. 16, 1971 that describes a power supply for use with a pacemaker wherein the power generator utilizes fluid pressure derived from the muscular contractions of the heart; U.S. Pat. No. 3,835,864 titled Intra-Cardiac Stimulator, issued Sep. 17, 1974 that describes a stimulator for intra-cardiac use that generates electricity by using magnetic induction or may a piezoelectric effect; and U.S. Pat. No. 3,835,864 titled High efficiency vibration energy harvester, issued Jan. 10, 2006 that describes an energy harvester system. These publications are hereby incorporated by reference into this disclosure for all purposes.

There is a long-felt need for an energy generating, charging and storage system suitable for use with an active implanted medical device, such as a pacemaker or defibrillator, which has the following advantageous characteristics: (1) Longer life: increase in time to a device's power depletion by about 50% to 100%, e.g. pacemaker battery life increases from 5 to 7.5 or to 10 years or more. (2) Superior reliability: lower failure rates leading to lower incidences of re-operation. (3) Lower total cost of ownership: reduction in total cost of implantation (including follow-up procedures). (4) Maintenance-free use. (5) Continuous charging with no need for the patient or physician to take active measures to charge the device. In particular, it would be highly desirable to provide a power generation system that was powered by the physical, chemical, or physiological activity of the subject into which the device was implanted. (6) Rapid charging. (7) Consistent power output and current generation. Additionally the energy generating system must take up no more than the volume of current generators, and the implantation procedure must be simple and reasonably familiar to the surgeon. Also, the battery of such a system should provide a high cell voltage, long cycle life, high discharge rate capability, high charge rate capability, no memory effect, no gas evolution, non-toxic chemicals in the battery, high energy density, ability to shape the battery in various configurations, low self-discharge, proper state-of-charge indication, and improved reliability. The current invention provides devices that meet these needs.

### BRIEF DESCRIPTION OF THE INVENTION

The invention provides devices, systems, methods and kits for generating, charging and storing electrical energy that are suitable for use with implanted medical devices, such as a pacemakers and defibrillators. In certain preferred embodiments, the invention includes a generator component that provides continuous, automatic charging. In some embodiments, the invention provides a power generation system that is powered by the physical, chemical, or physiological activity of the subject into which the device was implanted, such as the haemodynamic forces of blood flow or by the beating of the heart. The generator may produce power in various ways, for example via electromagnetic induction or via a piezoelectric effect. In other embodiments, the invention includes batteries that are recharged from an external source of source of electromagnetic radiation, such as an optical, electrical, or magnetic source. The invention may be embodied in a number of ways, some of which may be briefly described as follows.

Preferred embodiments encompass a kinetic electrical generator that is fully implantable and biocompatible. Fully implantable means that the entire structure of the generator may be implanted into the body of a subject. The generator is used for powering an implanted medical device, and comprises a magnet and a conductor, and further comprising electrical leads adapted for electrical communication with the conductor and with the implanted medical device. To say that the leads are adapted for electrical communication with the device means that the design and structure of the leads is specifically contrived to facilitate such electrical communication. The magnet and the conductor are moveable in relation to each other, wherein, in use, when the magnet moves relative to the conductor, a current is induced in the conductor which is transmitted through the electrical leads to the implanted medical device.

In stating that that the leads are adapted for electrical communication with the device, it is meant that the device could be any suitable device or component of such device, including an energy storage element such as a battery.

Other embodiments encompass a generator as described above wherein the conductor is a coiled, defining an elongated lumen about a longitudinal axis, i.e., the conductor forms a long coil which may be disposed along the interior length of a tube, such as a catheter or similar structure. The outer tube is generally made of an insulating material. The magnet is disposed at least partially within the lumen, meaning that the magnet is either partially within the lumen at all times, or is in the lumen at least some of the time when in use. The magnet is movable through the lumen of the coiled conductor, and in use, the magnet does move through the lumen when the generator is moved approximately along the longitudinal axis.

Other embodiments encompass a generator as described above further comprising an eccentrically weighted cam attached to a shaft wherein the shaft is in mechanical communication with the magnet such that the movement of the can causes a concomitant movement of the magnet. The eccentrically weighted cam can be of any suitable structure, so along as it provides movement of the shaft (axle) when the device is moved. One or more gears may be provided that mechanically connects the shaft and the magnet. Such gears may amplify the movement of the magnet.

In preferred embodiments the magnet is spherical or elongated, for example, roughly tubular or cylindrical.

The spherical magnet may be enclosed in a tubular compartment having a first end and a second end. In some embodiments, each end is enclosed by a wall and wherein the interior surface of each wall comprises a deflecting element adapted to repel the spherical magnet when the spherical magnet impinges against the deflecting element. The deflecting element can be selected from the group consisting of: a biased spring, an elastic buffer, and a magnet. The deflecting element may additionally incorporate a variable-gap capacitor or a piezoelectric material.

Other embodiments comprise a plurality of individual tubular compartments set end to end, each separated from the adjacent compartment by a wall, each containing at least one spherical magnet. See FIGS. 3 and 4.

In another alternate embodiment, the in generator described above, the conductor is movable and the magnet remains stationary in use.

In any of the embodiments, the generator may have, for example a largest dimension of not more than 5, 10, 15, 20, 30, 40, 50, 70 or 100 mm.

In any of the embodiments, the generator may produce an average power output of between the 40 μW and 1000 μW. Average power is the power output measured under actual or simulated use conditions over a period of, for example, an hour to several days.

In any of the embodiments, the generator may have a volume of between 0.25 cc and 50 cc, for example, up to 10, 20, 30, 40 or 50 cc.

Another alternate embodiment is a kinetic electrical generator that is fully implantable and biocompatible, for powering an implanted medical device, the generator comprising a variable distance capacitor mechanically connected to a sprung counterweight, wherein, when the sprung counterweight is moved, the a variable distance capacitor is compressed, thereby generating a current; and further comprising electrical leads adapted for electrical communication with variable distance capacitor and with the implanted medical device.

The invention also encompasses a method for powering an implanted medical device, the method comprising providing a kinetic electrical generator as described herein and electrically connecting the generator via the electric leads to the medical device; then implanting the medical device at a desired location; then implanting the generator at a desired location; and then causing the generator to be moved, thereby generating electricity to power the implanted medical device. The generator may be implanted in the proximity of the heart wall, such as near enough to the heart so that the beating of the heart will cause the generator to be moved. It may, for example, be attached to the myocardium or pericardium, placed within the myocardium or pericardium, upon the surface of the myocardium or pericardium, and thereby be subjected to regular pulsating movements produced by the beating of the heart, wherein the movements have a frequency of between bout 0.5 Hz to about 2 Hz, thereby generating electrical power in the range of about 40 μW and 200 μW. Alternatively the generator may be placed within the vicinity of the lung or other organ that moves with regularity.

The invention also encompasses a kit comprising: the generator as described herein, and an implantable medical device selected from: (a) a pacemaker, (b) a defibrillator, (c) a left ventricular assist devices, (d) a muscle stimulator, (e) a neurological stimulator, (f) a cochlear implant, (g) a monitoring device, and (h) a drug pump.

### BRIEF DESCRIPTION OF THE FIGURES

FIG. 1 is a schematic drawing showing a cut-away drawing of the charger (1) placed above the implanted device (pacemaker) (3). The charger is essentially a hollow roughly disc-shaped capsule containing multiple wire loops (2) running around the inside wall of the capsule. The charger is placed in proximity with the pacemaker such that the charger is placed against the skin, outside the patient, with the pacemaker lying just below the skin. A current is passed through the wire loops of the charger to produce an electromagnetic field. Alternating or varying the current produces a changing magnetic flux that radiated from the charger and penetrates the skin, such that the lines of flux intersect with and cut through the internal wire loops (4) of the pacemaker. This flux cutting induces a current in the internal wire loops (4) of the pacemaker which is used to charge internal batteries, or to provide power directly to one or more electrical components of the pacemaker.

FIG. 2 is a schematic drawing that shows three embodiments of kinetic charger systems: a rotating mass charger (6); a moving magnet charger (12), and a variable capacitor charger (13). Each charger is shown attached to a catheter (9). The catheter is electrically connected to the lead (12) of a pacemaker (17). The rotating mass charger (6) comprises a mass (7) that rotates about the axle of a micro-generator (8). The moving magnet charger (12) includes a magnet (11) that moves (slides) through a wire coil (10), inducing current in the coil. The variable capacitor charger (13) uses a mass placed on a spring (14) to sequentially compress and release a variable distance capacitor (15), thereby generating an electric current.

FIG. 3 is a schematic drawing of variation of a moving magnet-type generator (18) built into a catheter structure (19) comprising a plurality of individual magnetic spheres (20) each disposed within an elongated wire coil (22) that runs longitudinally through the catheter along the inside of the insulated catheter wall (21). 3A shows an expanded view of a single sphere.

FIG. 4 is a schematic drawing of an embodiment of a moving magnet-type generator showing a single closed generating unit (27) comprising a magnetic sphere (23) slidably and/or rollably disposed within an elongated hollow cylinder having an insulated casing (26) outside of which is wound a wire coil (24). A spring (26), is placed at each end of the interior of the cylinder so as to deflect the sphere which bounces off the spring, moving through the cylinder so as to induce an electric current in the exterior wire coil.

FIG. 5 is a schematic drawing showing a variable distance capacitor made from a “concertina” arrangement of aluminium-evaporated polyester film between two acrylic boards. In use, the capacitor generates an electric charge when compressed and released.

FIG. 6 is a drawing showing the components of a charging mechanism using an oscillating weight used to move a magnet and a coil relative to each other.

### DETAILED DESCRIPTION OF THE INVENTION

The present invention encompasses devices and systems for generating, charging and storing electrical energy. The devices and systems of the invention are biocompatible and are suitable for use with active implanted medical devices, such as a pacemakers and defibrillators, and also with ventricular assist devices, muscle stimulators, neurological stimulators, cochlear implants, monitoring devices, and drug pumps.

With relationship to the invention, biocompatibility means that the device or material is relatively inert in a biological context, so that when implanted the device or material does not react with biological material in a detrimental way

Certain of the embodiments of the invention include a generator component that provides continuous, automatic charging. In various embodiments, the generator of the device generates electricity with no need for the patient or physician to take active measures to charge the device, in particular, the invention provides a power generation system that is powered by the physical, chemical, or physiological activity of the subject into which the device was implanted. Specifically, certain embodiments of the invention provide a power generation system that is powered by heat differentials, physiological pressures, flows and movements, such as the haemodynamic forces of blood flow or by muscular contractions and movements, such as those produced by the beating of the heart myocardium.

The generator may be incorporated and integrated into the structure of an implanted device, such as a pacemaker, or it may be remote from the pacemaker, and attached functionally, in electrical communication via a conductor (a lead).

In one embodiment, the invention provides an automatic, continuous electrical generator that is disposed within a catheter that may be positioned as desired, within an area of movement or muscular activity, such as adjacent to the heart. In certain preferred embodiments, the generator may produce electrical power using various means, such as: electromagnetic induction, or by heat differential, or in another, via a piezoelectric effect.

Energy produced by the generator of the invention may be stored as electrical potential energy, usually using a chemical battery. Many such devices are well known in the art. Batteries used with the invention may be rechargeable or non-rechargeable. Electrical energy may also be stored in a capacitor. In certain embodiments, the device may include both a battery and a capacitor wherein one functions as a back-up to the other. Alternatively the device may also include a non-rechargeable battery to act as a back-up source of energy in case of failure of another energy storage component.

### EMBODIMENTS OF THE INVENTION

The invention encompasses various embodiments that employ generator components that generate energy using different principles, as set out below.

### 1. Kinetic Charging

### Electromagnetic Generators

Mechanical and kinetic energy is converted by electromagnetic induction into useable or storable electrical energy to be used to power the device (e.g., pacemaker or ICD). The mechanical energy or motion may come from a variety of sources, for example the heart, which provides continuous motion, reliability, and proximity to the rest of the pacing and/or defibrillation apparatus. Contraction of the heart muscle causes relative motion between a magnetized body(s) and electrically conducting elements(s) such as an induction coil. The relative motion between the magnet and the conductor will induce a current to flow in the conductor. The motion may be translation, rotation, flexure, or any combination of such.

The electrical conductor(s) (wires) may be arranged in loops or assume other forms to collect the most magnetic flux. This wire may be wrapped at various pitch angles around a tube through which the magnet moves in, or coiled above or below the end of the magnet's travel. Benefits may be obtained by using magnetic, ferromagnetic, paramagnetic, or non-magnetic materials to make the tube. Any of these materials may also be held within the loops of coils, or in a location where they may come in contact with the magnet, or the poles of the magnet, during its travel, or at the completion of its travel to complete a magnetic flux circuit.

Current generated by a kinetic generator can be stored or used immediately. A variety of circuits, storage devices, batteries etc may be employed to collect and/or store the energy.

In certain kinetic embodiments the motion of a magnet may be constrained by a tube or race that may contain the conductive wires. This guide can be straight, curved, or even a ring depending in the optimization of the system. It may be structured to encourage rotation, translation, or a combination of the two as a result of inertial forces. The tube may be filled with wet or dry lubricant, MR fluid, vacuum, or air to effect the response of the system or the dynamics between multiple masses.

The ends of the tube may contain springs or other magnets to “bounce” the magnet to travel to the other side, and/or possible reverse the direction of spin. The springs may be tuned such that the system exhibits resonance. The springs themselves may be electrically conducting wires capable of capturing flux. The springs may include variable-gap capacitors or piezoelectric materials capable of producing voltage when stressed.

In each various embodiments of the invention the magnet(s) or wire(s) maybe as small as MEMS or Nanometer-sized structures.

One advantage of kinetic charging is the potential for passive energy scavenging. Energy can be collected without any demands upon the patient or medical practitioners, and the energy may be provided at a rate sufficient to power the pacing device for as long as the heart continues to beat. The generator of the present invention can provide enough energy to power an implanted device by harvesting less than 1% of the available energy at the catheter tip. In a preferred embodiment, the generator of the invention produces sufficient electricity to power the implanted device to which it is coupled. In most instances, 40 μW is sufficient, although power generation of up to 1 mW is obtainable. In certain embodiments, even larger amounts of power may be produced, for example by using multiple devices or devices with multiple units. This allows for a much smaller battery pack than traditional technology, potentially reducing the overall size of the device by roughly one third to one half or more. For example, a typical commercial pacemaker with a volume of 16 milliliters may be reduced in overall size to between about 11 ml and 8 ml. A defibrillator of 50 ml could be reduced in size to between about 35 ml to 25 ml.

In a preferred embodiment, a kinetic generator is integrated into one or more lead(s) which are fixed to the ventricle wall. By placing the kinetic generator on the heart wall the generator is subjected to nearly continuous oscillations on the order of 1 Hz corresponding with a pulse rate of 60 beats per minute. A mechanically tuned system could take advantage of this consistent rhythm and be designed to take advantage of mechanical resonance to amplify the vibration. Resonance is a well understood phenomenon, and the ability to design the generator of the invention such that its resonant frequency is at or close to that of the physical impulse that drives it should be a matter of routine design.

For intravenous implantation, for example into the subclavian vein, may be done using standard practices which are well known in the field. In the standard procedure the leads are placed through the subclavian vein and threaded through the vein into the right side of the heart. Depending on how many leads the pacemaker has, one is implanted into the Apex (tip) of the heart which is the right ventricle. They are then secured (often by a screwing action) to the endocardium. The other lead can be implanted into the right atrium (usually the medial wall), and if it is a biventricular pacer, a third leads is snaked into the coronary sinus onto the left side of the heart. The generator may be approximately cylindrical and the outer diameter should not exceed 4 mm. The length is somewhat less constrained, as long as the device is not so large that it interferes with cardiovascular performance or prevents implantation.

Kinetic generators of the invention can be engineered to provide about 40 μW for an indefinite period of operation, sufficient to power a pacemaker or defibrillator. In some embodiments, the magnetic generators of the invention can produce energy of as much as 1 mW (see Mitcheson et al., “Architectures for Vibration-Driven Micropower Generators,” J. Microelectromechanical Systems, vol. 13, no. 3, 2004, pp. 429-440).

Average power produced by a generator of the invention over a 24 hour period can be from about 10 μW to about 1000 μW, for example, at least 30 μW, at least 40 μW, at least 60 μW, at least 100 μW, at least 150 μW, at least 200 μW, at least 300 μW, or at least 500 μW on average.

A single generator unit may be used, or in certain embodiments, a plurality of such units may be used to provide the desired power. Different generator types may be combined in a single device.

The generator unit will be positioned at or close to the tip of a cardiac catheter lead, but this need not be the case, and positioning will be done as appropriate taking into account the degree of movement that will be imparted to the generator at any particular location, and the difficulty and dangers inherent with implantation at a particular location.

There are several embodiments that may be used for a kinetic charging system. See FIG. 2 that shows a rotating mass embodiment, a moving magnet embodiment, and a variable capacitor embodiment.

### (i) Moving Mass Embodiment

A preferred embodiment produces current by electromagnetic induction. The invention encompasses both moving-magnet and moving coil embodiments. In either case the relative motion provides flux-cutting which induces an electrical current in the coil. Relative motion between a magnet and a wire induces electrical current in the wire.

A translational or rotational mass can be used to move, oscillate or spin a magnet relative to a coil of wire similar to the micro-generators used to generate electricity in watches, for example those manufactured by Seiko (See FIG. 6). A current is induced in the wires that are in electrical contact with one or more components of an implanted device, either to provide power directly, or to be stored in a storage device such as a chemical battery.

Such moving mass generators can provide an almost constant power sufficient to power a pacemaker or defibrillator for indefinite operation.

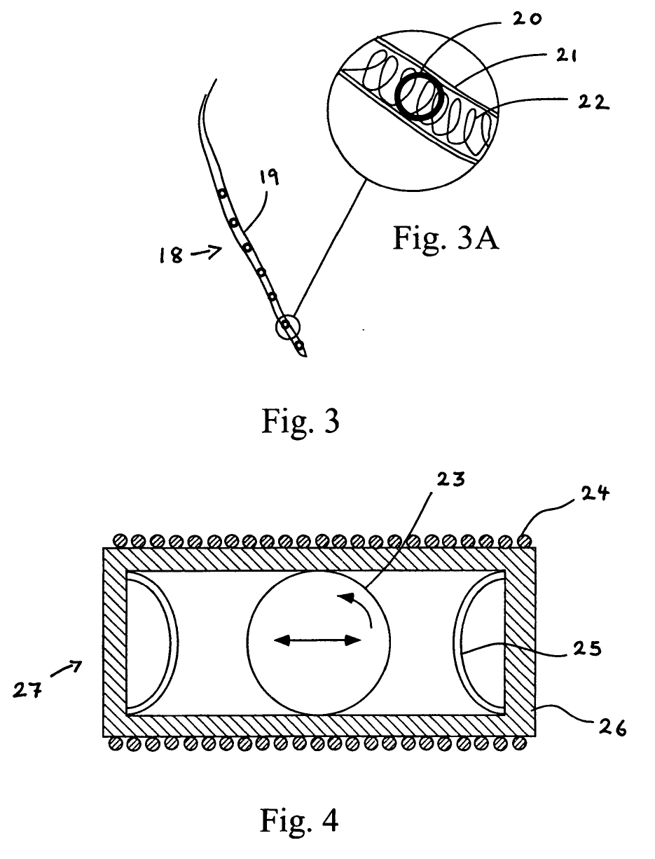

One embodiment, shown in FIGS. 3 and 4, utilizes a magnetic sphere that moves back and forth within a coiled conductor, inducing a current in the conductor. The conductor is in electrical communication with an implanted device. FIG. 3 shows a moving magnet-type generator built into a catheter structure (19) with a number of individual magnetic spheres (20) inside an elongated wire coil (22). The magnetic balls move by rolling and sliding within the length of the wire coil inducing a current that is then transmitted to an attached implanted device, such as a defibrillator. FIG. 3A shows an expanded view of a single sphere. FIG. 4 shows a single closed generating unit (27) comprising a magnetic sphere (23) that slide and/or rolls within an elongated hollow cylinder having an insulated casing (26). A wire coil (24) is wound around the casing and is in electrical contact with an implanted device. Springs (26) are present at each end of the interior of the cylinder so as to deflect the sphere which bounces off the spring, moving through the cylinder so as to induce an electric current in the exterior wire coil.

In a related embodiment the cylinder may be fitted with a magnet at each end such that when the magnet reaches the end of the coil, it is repelled back to the other end setting up an oscillatory motion that could generate more energy.

In other embodiments the magnet need not be spherical, and need not roll, but can be of any shape, for example it may be an elongated polyhedron or cylinder, a pill shape or an oval, rectangular, prism etc that is allowed to slide through a wire coil. The term “coil” is not used to imply a circular structure. The wire coil may be of any shape and may simply be produced by winding a wire conductor onto an armature of a desired shape and dimension. Generally the magnet will be designed to fit fairly closely within the wire coil to provide the maximum flux density, and therefore the maximum current.

There are also commercial integrated circuits in which a matrix of small magnets “flapping” within wire loops has been used to generate electric current. One such manufacturer is Ferro Solutions.

### (ii) Variable Distance Capacitor Embodiment

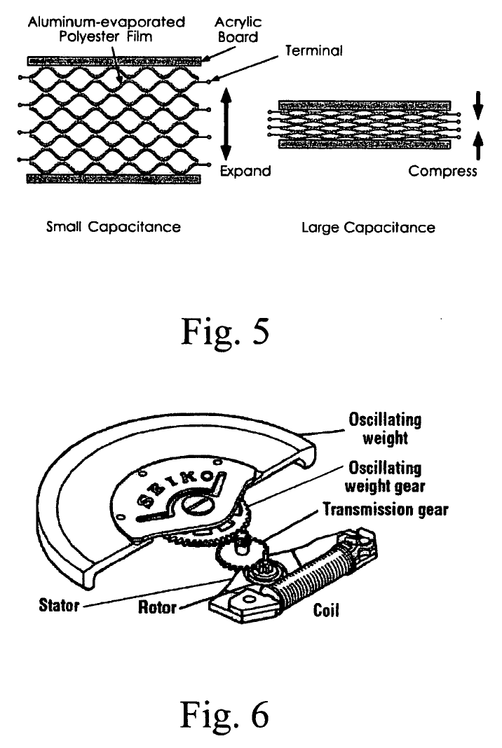

Another embodiment that employs kinetic charging is a device that employs a variable distance capacitor (also referred to as a variable capacitor or VC) instead of the magnetic micro-generator. (See FIG. 5). A variable distance capacitor can be implemented in a similar way as the other kinetic “shakers” but with less discrete moving parts. It can take advantage of the motion of the heart, being tuned with a resonant frequency of the pulse, or have the motion of the heart or other force to actuate the plates, which contract and expand, thereby producing an electric current. Such a capacitor could also be powered by pressure changes rather than using the acceleration of the heart. Certain researchers have found that the mean power generated using a prototype VC in a dog study was 36 μW over a span of 2 hours. See Ryoichi Tashiro et al., “Development of an electrostatic generator for a cardiac pacemaker that harnesses the ventricular wall motion” J Artif Organs (2002) 5:239-245. Human anatomy allows for a larger VC to be used, hence higher power could be expected.

### (iii) Piezoelectric Embodiment

An alternative embodiment is to employ piezoelectric technology. Piezoelectric elements convert force or strain into electrical potential. Piezo elements can be used to harvest energy when subject to indirect (inertial) forces or when subject to direct forces caused by the heart contraction.

One piezoelectric embodiment employs a layer of piezo wire spanning the length of the entire lead. Such wire can be obtained commercially (e.g., Ormal Vibetek Piezo™ wire). The piezo wire may, for example have a thickness of about 2.7 mm including insulation. One embodiment employs a novel lead wire made with a layer of polyvynldifluride (PVDF) piezo material. As the heart beats, the piezo is subject to strain as the wire “flops around,” and electricity is generated away and transmitted to the device or battery.

In one embodiment, the structure of such a wire would have traditional lead components at the core, surrounded by an insulator material. The insulator material would be surrounded by conductive material, which would then be surrounded by a PVDF layer, which in turn would be surrounded by another layer of conductive material, which would finally be surrounded by an outer insulating layer, such as a silicone jacket. The piezo material sandwiched between the conducting layers would produce an electric charge that would produce a current that would flow through the conductors. The conductors would be in electrical contact with one or more components of an implanted device, such as with the storage device (generally a chemical battery).

Another approach can combine elements of the above concepts in attempt to achieve higher efficiency levels, and build redundancy into the system to compensate for a component failure if one should occur. A micro-generator or variable capacitor element could be placed on the end of a piezoelectric wire/lead combination.

### 2. Optical Charging

This concept involves charging an implanted electro-cardio device's internal battery by transmitting optical power through the skin into an array of photovoltaic cells implanted beneath the surface of the skin. Power in the form of near-infrared light may be beamed from an optical power source outside the body onto the photovoltaic cell array, which is embedded under the skin. The power received by these cells is then used to charge or recharge the implanted device's internal rechargeable battery. The photovoltaic cells (photo collector) are in electrical connection via an electrical conduit (lead) with a battery. The battery is in electrical connection with the electrical circuitry or the pacemaker or other device.

The power source may be in the form of a high-power near-infrared-laser diode, and the photovoltaic cell array power receiver may consist of photodiodes. Near-infrared light may be utilized due to its low invasiveness to tissues, since the optical power would pass directly through the skin. Unlike the radio frequency waves used in electromagnetic inductive charging techniques, light does not interfere with operation of the implanted device. The photovoltaic cell array can be packaged for biocompatibility and hermetically sealed. The patient or physician may charge the device in predetermined intervals simply by placing the light source close to the surface of the skin, above the photo collector, for a pre-determined time.

Near-infrared light is particularly suitable for such a device, but other wavelengths of light and other types of electromagnetic radiated power may also be used.

There are two embodiments in which the optical concept can be implemented: the photovoltaic cell array can be either packaged into (on the surface of) the implanted device, or can be embedded in a separate part of the device and connected to the implanted device by a wire. In addition, the power transmission level of the optical transmitter and the area of the photovoltaic cell array can be altered to increase or decrease the amount of power delivered and received, provided the irradiation or heat does not cause damage to human skin and tissue, and the size is not prohibitive for implantation in the human body.

### 3. Thermoelectric Charging

Thermoelectric power can be utilized to power an implanted electro-cardio device, or charge its internal battery through the use of thermoelectric materials that produce an electrical current in the presence of a temperature gradient. Thermoelectric materials are essentially semiconductors which consist of pairs of p-type and n-type towers connected electrically in series, which produce electric current through the Seebek Effect. By inserting a layer of thermoelectric material between two media of different temperatures to leverage the human body's natural thermal processes, an electrical current may be produced in the material, which is then harnessed by the implanted device. The thickness of the thermoelectric material is the distance between the hotter side and the colder side, and may be, for example, about 3 mm (See M. Wiener, S. Cooper, “Nanotechnology Based Biothermal Materials For Implantable Devices and Other Applications,” Ind. Biotech., vol. 1, no. 3, pp. 194-195, fall 2005).

A thermoelectric generator may be constructed as follows. A sheet of thermoelectric material is sandwiched between the skin and either the device casing, muscle, or external environment, and surrounding the edges of the material with insulating material (such as a ceramic or a hydrocarbon polymer material) to preserve the temperature gradient and optimize heat flow. Power can be generated to be delivered to the device battery or to the device directly via conductors electrically connecting to the thermoelectric generator and the device. If a battery is used, then the thermoelectric generator is placed in electrical contact with the battery via a conductor, and simply charges the battery in the usual way. Without the use of a rechargeable battery, an implanted device relying solely on the use of thermoelectric materials for power generation can be continually and perpetually powered, with the lifetime of the device limited only by the patient's lifetime, disregarding any need to replace the device due to malfunction or degradation.

The thermoelectric charging system may be implemented in a variety of ways. The thermoelectric sheet can be placed within a part of the body that produces a high temperature differential, for example between a superficial blood vessel or capillary bed and the skin surface, and far from the device itself, with a wire running to the implanted device. Also, the material can be placed externally outside the human body, or integrated onto the casing of the device, utilizing the temperature gradient between the skin and underlying tissue or open space in the casing. In addition, the area of the material can be adjusted to generate different amounts of current, as desired.

The thermoelectric charging system as described using current thermoelectric materials can produce enough power to indefinitely power a pacemaker device without the use of an on-board battery, with the lifetime of the pacemaker constrained only by device malfunction or degradation. In one preferred embodiment, the generator supplies electric current to a capacitor coupled to a non-rechargeable battery. ICDs can also utilize this power delivery mechanism to recharge a battery until the battery is depleted and can no longer sustain the ICD. This provides a very significant improvement in lifetime of the ICD. Indeed, using newer battery chemistry, such as that available commercially from Quallion Corporation, it is anticipated that an ICD could easily have a 10 year life or more. An ICD with a such batteries, for example a Polysiloxane polymer electrolyte lithium battery, employing the thermoelectric charging system of the invention will have an average working life-span of greater than 5 years, for example greater than 7 years, greater than 10 years, greater than 13 years or even greater than 15 years.

There are various available rechargeable battery technologies that can be used with the invention. Of the common rechargeable battery technology, lithium ion batteries have the highest energy density (about ⅔ that of current non-rechargeable pacemaker batteries). Nickel metal hydride batteries have an energy density that is roughly ⅓ that of current non-rechargeable batteries. Accordingly, rechargeable batteries will not last as long as non-rechargeable batteries using current technology on a single charge. Additionally, the lifespan of common rechargeable batteries is limited. A lithium ion battery has a lifespan of approximately 5 years, while nickel metal hydride has a lifespan on the order of 10 years.

### 4. Direct Plug-In

This embodiment encompasses a fully implantable device that can be charged via an external power source by direct electrical conductive contact with the implanted device. The charging mechanism is implemented in a manner that resists infection or other complications. The is be charged by transdermally establishing a direct connection to the device's power source's terminals, much like how a power plug is inserted into a standard wall electrical socket.

One embodiment is to “inject” leads in a similar fashion as syringe needle injections. These relatively small incisions will reduce the chance of infection a negligible value; most sterile needle injections carry little risk of infection. In order to reduce the incidence of applying a voltage potential across body tissue, there will be a need for the contacts on the implanted device to be insulated from body tissue by covering the leads with an insulating material.

Another embodiment consists of two large contacts placed on the surface of the pacemaker, and insulator placed over them. A special plate designed to approximate the size of the pacemaker, is used to easier align the charging leads with the pacemaker externally and allow for easy charging. The pacemaker has two insulated contacts on its surface, through which the leads will be inserted. A metallic device fits over the pacemaker. This device will be used to fit over the shape of the pacemaker transdermally and therefore allows the physician or nurse to more effectively guide the charging needles to the pacemaker charging contacts. These two leads can be bundled or entwined into one integrated wire, much like a coaxial cable, and thus require only one connection instead of two into the implanted device.

### 5. Wireless Induction

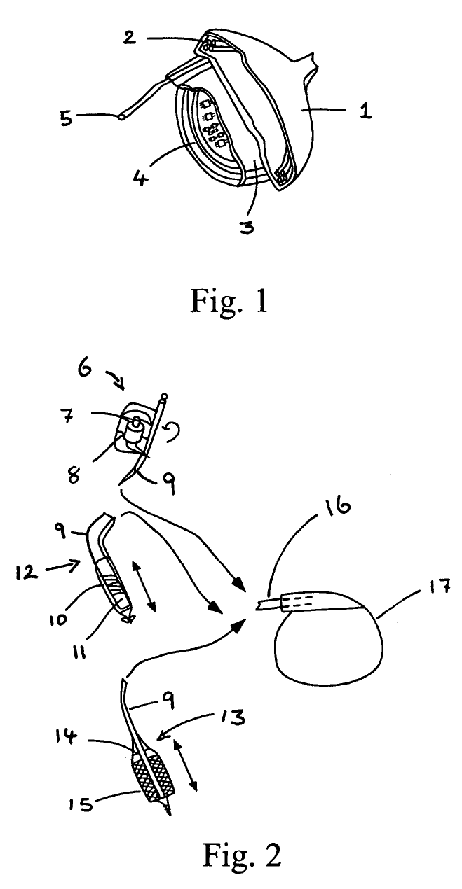

This embodiment involves inductively charging the implanted device in a wireless manner using electromagnetic force at radio frequencies. A current is run through the power supplying coil, which induces a current in the coil encased within the implanted device when placed near to each other. Thus, current can be generated and delivered to the power supply without any physical connections to the power supply. See FIG. 1 that shows a cut-away drawing of the charger (1) placed above the implanted device (pacemaker) (3). The charger is essentially a hollow roughly disc-shaped capsule containing multiple wire loops (2) running around the inside wall of the capsule. The charger is placed in proximity with the pacemaker such that the charger is placed against the skin, outside the patient, with the pacemaker lying just below the skin. A current is passed through the wire loops of the charger to produce an electromagnetic field. Alternating or varying the current produces a changing magnetic flux that radiated from the charger and penetrates the skin, such that the lines of flux intersect with and cut through the internal wire loops (4) of the pacemaker. This flux cutting induces a current in the internal wire loops (4) of the pacemaker which is used to charge internal batteries, or to provide power directly to one or more electrical components of the pacemaker.

The size of the coils and the number of turns in the coil determines the amount of power delivered. With heat restrictions, and optimal power delivery amount can be determined using standard calculations.

### 6. Pressure Energy: Piston-Diaphragm

Variations in blood pressure between the systole and diastole cause displacement of a membrane, diaphragm, piston, or other type of transducer that can be connected to an energy conversion element.

### Other Embodiments of the Invention

The invention also encompasses a method for powering an implanted medical device, the method comprising: (1) providing a kinetic electrical generator that is fully implantable and biocompatible, for powering an implanted medical device, the generator comprising a magnet and a conductor; and further comprising electrical leads adapted for electrical communication with the conductor and with the implanted medical device; wherein the magnet and the conductor are moveable in relation to each other; wherein the conductor is a coiled, defining an elongated lumen about a longitudinal axis, and the magnet is disposed at least partially within the lumen, and is movable through the lumen of the coiled conductor, and wherein, in use, the magnet does move through the lumen when the generator is moved approximately along the longitudinal axis; (2) electrically connecting the generator via the electric leads to the medical device; (3) implanting the medical device at a desired location; (4) implanting the generator at a desired location; (5) causing the generator to be moved, thereby generating electricity to power the implanted medical device.

Using the above methods, the generator may be implanted in the proximity of the heart wall and thereby be subjected to regular pulsating movements produced by the beating of the heart, wherein the movements have a frequency of between bout 0.5 Hz to about 2 Hz, thereby generating electrical power in the range of about 40 μW and 200 μW.

The invention also encompasses a kit comprising: (1) a kinetic electrical generator that is fully implantable and biocompatible, for powering an implanted medical device, the generator comprising a magnet and a conductor; and further comprising electrical leads adapted for electrical communication with the conductor and with the implanted medical device; wherein the magnet and the conductor are moveable in relation to each other; wherein the conductor is a coiled, defining an elongated lumen about a longitudinal axis, and the magnet is disposed at least partially within the lumen, and is movable through the lumen of the coiled conductor, and wherein, in use, the magnet does move through the lumen when the generator is moved approximately along the longitudinal axis; and (2) an implantable medical device selected from the group consisting of: (a) a pacemaker, (b) a defibrillator, (c) a left ventricular assist devices, (d) a muscle stimulator, (e) a neurological stimulator, (f) a cochlear implant, (g) a monitoring device, and (h) a drug pump.

### GENERAL REPRESENTATIONS CONCERNING THE DISCLOSURE

The embodiments disclosed in this document are illustrative and exemplary and are not meant to limit the invention. Other embodiments can be utilized and structural changes can be made without departing from the scope of the claims of the present invention. As used herein and in the appended claims, the singular forms “a”, “an”, and “the” include plural reference unless the context clearly dictates otherwise. Thus, for example, a reference to “a part” includes a plurality of such parts, and so forth.

In the present disclosure reference is made to particular features of the invention. It is to be understood that the disclosure of the invention in this specification includes all appropriate combinations of such particular features. For example, where a particular feature is disclosed in the context of a particular embodiment or a particular claim, that feature can also be used, to the extent appropriate, in the context of other particular embodiments and claims, and in the invention generally.

The embodiments disclosed in this document are illustrative and exemplary and are not meant to limit the invention. Other embodiments can be utilized and structural changes can be made without departing from the scope of the claims of the present invention. In the present disclosure, reference is made to particular features (including for example components, ingredients, elements, devices, apparatus, systems, groups, ranges, method steps, test results, etc). It is to be understood that the disclosure of the invention in this specification includes all possible combinations of such particular features.

As used herein and in the appended claims, the singular forms “a”, “an”, and “the” include plural reference unless the context clearly dictates otherwise. Thus, for example, a reference to “a part” includes a plurality of such parts, and so forth.

The term “comprises” and grammatical equivalents thereof are used herein to mean that, in addition to the features specifically identified, other features are optionally present. The term “at least” followed by a number is used herein to denote the start of a range beginning with that number (which may be a range having an upper limit or no upper limit, depending on the variable being defined). For example “at least 1” means 1 or more than 1, and “at least 80%” means 80% or more than 80%. The term “at most” followed by a number is used herein to denote the end of a range ending with that number (which may be a range having 1 or 0 as its lower limit or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number)”, this means a range whose lower limit is the first number and whose upper limit is the second number.

Where reference is made herein to a method comprising two or more defined steps, the defined steps can be carried out in any order or simultaneously (except where the context excludes that possibility), and the method can optionally include one or more other steps which are carried out before any of the defined steps, between two of the defined steps, or after all the defined steps (except where the context excludes that possibility). The numbers given herein should be construed with the latitude appropriate to their context and expression; for example, each number is subject to variation which depends on the accuracy with which it can be measured by methods conventionally used by those skilled in the art.

This specification incorporates by reference all documents referred to herein and all documents filed concurrently with this specification or filed previously in connection with this application, including but not limited to such documents which are open to public inspection with this specification.

### REFERENCES

Moss, A. Long QT Syndrome. Current Treatment Options in Cardiovascular Medicine 2000, 2:317-322
K. Murakawa, M. Kobayashi, O. Nakamura, and S. Kawata, “A wireless near-infrared energy system for medical implants,” IEEE Eng. Med. Biol. Mag., vol. 18, pp. 70-72, November/December 1999.
K. Goto, T. Nakagawa, O. Nakamura, S. Kawata, “An implantable power supply with an optically rechargeable lithium battery,” IEEE Trans. Biomed. Eng., vol. 48, no. 7, pp. 830-833, July 2001.
K. Goto et al., “Feasibility of the automatic generating system(ags) for quartz watches as a leadless pacemaker power source,” IEEE Trans. Biomed. Eng., vol. 20, no. 1, pp. 417-419, 1998.
Joseph A. Paradiso, Thad Starner. “Energy Scavenging for Mobile and Wireless Electronics” PERVASIVEcomputing Published by the IEEE CS and IEEE ComSoc 1536-1268/05 IEEE 2005.
Sodano et al., “A review of power harvesting from vibration using piezoelectric materials,” Shock and Vibration Digest, Vol. 36, No. 3, May 2004 pp. 197-205
Roundy, S. “Energy Scavenging for Wireless Sensor Nodes with a Focus on Vibration-to-Electricity Conversion,” 2006.

1. A kinetic electrical generator that is fully implantable and biocompatible, for powering an implanted medical device, the generator comprising a magnet and a conductor; and further comprising electrical leads adapted for electrical communication with the conductor and with the implanted medical device; wherein the magnet and the conductor are moveable in relation to each other; wherein, in use, when the magnet moves relative to the conductor, a current is induced in the conductor which is transmitted through the electrical leads to the implanted medical device.

2. The generator of claim 1 wherein the conductor is a coiled, defining an elongated lumen about a longitudinal axis, and the magnet is disposed at least partially within the lumen, and is movable through the lumen of the coiled conductor, and wherein, in use, the magnet does move through the lumen when the generator is moved approximately along the longitudinal axis.

3. The generator of claim 2 further comprising an eccentrically weighted cam attached to a shaft wherein the shaft is in mechanical communication with the magnet such that the movement of the cam causes a concomitant movement of the magnet.

4. The generator of claim 3 further comprising one or more gears mechanically connecting the shaft and the magnet.

5. The generator of claim 2 wherein the magnet is spherical.

6. The generator of claim 5 wherein the spherical magnet is enclosed in a tubular compartment having a first end and a second end.

7. The generator of claim 6 wherein each end is enclosed by a wall and wherein the interior surface of each wall comprises a deflecting element adapted to repel the spherical magnet when the spherical magnet impinges against the deflecting element.

8. The generator of claim 7 wherein the deflecting element is selected from the group consisting of: a biased spring, an elastic buffer, and a magnet.

9. The generator of claim 8 wherein the deflecting element additionally incorporates a variable-gap capacitor or a piezoelectric material.

10. The generator of claim 2 wherein the magnet is an elongated magnet, wherein the elongated magnet is enclosed in a tubular compartment having a first end and a second end.

11. The generator of claim 10 wherein each end is enclosed by a wall and wherein the interior surface of each wall comprises a deflecting element adapted to repel the elongated magnet when the elongated magnet impinges against the deflecting element.

12. The generator of claim 6 comprising a plurality of individual tubular compartments set end to end, each separated from the adjacent compartment by a wall, each containing at least one spherical magnets.

13. The generator of claim 2 wherein the conductor movable and wherein the magnet remains stationary in use.

14. The generator of claim 2 having a largest dimension of not more than 20 mm.

15. The generator of claim 2 which in use produces an average power output of between the 40 μW and 1000 μW.

16. The generator of claim 2 having a volume of between 0.25 cc and 5 cc.

17. A kinetic electrical generator that is fully implantable and biocompatible, for powering an implanted medical device, the generator comprising a variable distance capacitor mechanically connected to a sprung counterweight, wherein, when the sprung counterweight is moved, the a variable distance capacitor is compressed, thereby generating a current; and further comprising electrical leads adapted for electrical communication with variable distance capacitor and with the implanted medical device.

18. The generator of claim 17 which in use produces an average power output of between the 40 μW and 1000 μW.

19. A method for powering an implanted medical device, the method comprising: (1) providing a kinetic electrical generator that is fully implantable and biocompatible, for powering an implanted medical device, the generator comprising a magnet and a conductor; and further comprising electrical leads adapted for electrical communication with the conductor and with the implanted medical device; wherein the magnet and the conductor are moveable in relation to each other; wherein the conductor is a coiled, defining an elongated lumen about a longitudinal axis, and the magnet is disposed at least partially within the lumen, and is movable through the lumen of the coiled conductor, and wherein, in use, the magnet does move through the lumen when the generator is moved approximately along the longitudinal axis; (2) electrically connecting the generator via the electric leads to the medical device; (3) implanting the medical device at a desired location; (4) implanting the generator at a desired location; (5) causing the generator to be moved, thereby generating electricity to power the implanted medical device.

20. The method of claim 19 comprising implanting the generator in the proximity of the heart wall and further comprising subjecting the generator to regular pulsating movements produced by the beating of the heart, wherein the movements have a frequency of between bout 0.5 Hz to about 2 Hz, thereby generating electrical power in the range of about 40 μW and 200 μW.
